(19) United States
(12) Patent Application Publication
(10) Pub. No.: US 2026/0216218 A1
(43) Pub. Date: Jul. 30, 2026

(54) CANNABINOID COMPOSITIONS, AND USES THEREOF IN TREATMENT OF NEURODEGENERATIVE DISEASES OR DISORDERS AND CANCERS

(71) Applicant: Biopharmaceutical Research Company, Castroville, CA (US)

(72) Inventors: Michael Hunter LAND, Kure Beach, NC (US); George Bennett HODGIN, Carmel Valley, CA (US); Brian Kent BRANDLEY, Fresno, CA (US); Lucas J. ZUMSTEIN, Marina, CA (US)

(73) Assignee: Biopharmaceutical Research Company, Castroville, CA (US)

(21) Appl. No.: 19/149,054

(22) PCT Filed: Jan. 19, 2024
(86) PCT No.: PCT/US2024/012229

§ 371 Date: Jul. 17, 2025

Related U.S. Application Data
(60) Provisional application No. 63/440,340, filed on Jan. 20, 2023.
(60) Provisional application No. 63/502,382, filed on May 15, 2023.
(60) Provisional application No. 63/614,420, filed on Dec. 22, 2023.

(51) Int. Cl. A61K 31/00; A61K 36/185; A61K 47/12

(52) U.S. Cl. CPC A61K 31/658; A61K 36/3482; A61K 47/12

(57) ABSTRACT

Cannabinoid compositions formulated for use in the treatment of neurodegenerative diseases and conditions, and cancer are disclosed. The treatment methods herein may be targeting the microtubule associated protein Tau. The compositions include cannabidiol (CBD), cannabigerol (CBG), and tetrahydrocannabinol (THC) as the major components therein, which may be isolated, purified or extracted from cultivars and blended together. The compositions include CBD, CBG and THC in certain ratios, formulated for the use in the treatment of neurodegenerative diseases and conditions, and cancer.

### CROSS-REFERENCE TO RELATED APPLICATIONS

This application claims priority to U.S. Provisional Patent Application Nos. 63/440,340, filed Jan. 20, 2023, 63/502,382, filed May 15, 2023, and 63/614,420, filed Dec. 22, 2023, each of which is incorporated herein by reference in their entirety.

### FIELD

The present disclosure relates generally to cannabinoids for use in treatment of neurodegenerative diseases or disorders, and more specifically to compositions comprising a combination of cannabidiol (CBD), cannabigerol (CBG), and tetrahydrocannabinol (THC) for treatment of neurodegenerative diseases and conditions, as well as cancers.

### BACKGROUND

Cannabinoids encompass several classes of compounds found in the cannabis plant. Cannabinoids have been explored for various medical uses, from glaucoma to cancer. For example, there is some evidence that cannabinoids can improve nausea and vomiting after chemotherapy. See Can Fam Physician. 2018 February; 64(2): e78-e94.

Cannabinoids have biological effects in the central and peripheral nervous systems, including the regulation of neuronal homeostasis and survival, which may be the result of the combination of a myriad of effects addressed to preserve, rescue, repair, and/or replace neurons, and also glial cells against multiple insults that may potentially damage these cells. See Neurotherapeutics. 2015 October; 12(4): 793-806. Cannabinoids have been explored as potential therapies for treating acute and chronic neurodegenerative pathological conditions. However, there remains an unmet need in the art for efficacious treatments of neurodegenerative diseases and conditions using cannabinoids.

### BRIEF SUMMARY

In some aspects, provided is a cannabinoid composition comprising cannabidiol (CBD), cannabigerol (CBG), and tetrahydrocannabinol (THC) present as a major component therein. In certain embodiments, the CBD, CBG and/or THC may be provided as: (i) one or more botanical drug substances (“BDS”); (ii) one or more extracts from cannabis plants (“extracts”); (iii) one or more extracts from cannabis plants blended with additional sources of CBD, CBG and/or THC (“blended extracts”); (iv) purified CBD, CBG and/or THC, e.g., obtained from purifying the extracts or blended extracts; or (v) isolates of CBD, CBG and/or THC. In some variations, the cannabinoid composition further comprises other cannabinoids and non-cannabinoids (e.g., terpenes) formulated in a vehicle (e.g., a lipid vehicle) to yield the final product composition that may be administered to a subject in need thereof. Such other cannabinoids and non-cannabinoids (e.g., terpenes) are present from the source from which the composition is obtained.

In other aspects, provided is a method for treating a neurodegenerative disease or condition, or a cancer in a subject in need thereof, comprising administering to the subject a therapeutically effective amount of any of the cannabinoid compositions described herein. In certain aspects, provided is a method for treating a tau-mediated neurodegenerative disease or condition, or a tau-mediated cancer in a subject in need thereof, comprising administering to the subject a therapeutically effective amount of any of the cannabinoid compositions described herein.

### DETAILED DESCRIPTION

The following description sets forth exemplary compositions, methods, parameters and the like. It should be recognized, however, that such description is not intended as a limitation on the scope of the present disclosure but is instead provided as a description of exemplary embodiments.

Cannabinoids are compounds structurally or pharmacologically related to the constituents of the cannabis plant or to the endogenous agonists (endocannabinoids) of the cannabinoid receptors CB1 or CB2. Cannabinoids may be naturally derived from cannabis plants or synthetically derived. Cannabis plants comprise a highly complex mixture of compounds, and hundreds of such compounds have been identified.

Traditionally, crude extracts from cannabis plants containing CBD have been used by patients suffering from various diseases and disorders. However, such crude products are generally unsuitable for use in pharmaceutical formulations. Those seeking to prepare more consistent CBD formulations for use in treating diseases or disorders have made an effort to either prepare CBD synthetically or attempt to remove all compounds other than CBD, particularly psychoactive compounds such as THC, from plant derived cannabinoids.

The present invention encompasses the surprising discovery that particular compositions comprising CBD in combination with CBG and THC have an improved therapeutic efficacy for treating tau-mediated diseases and conditions. The cannabinoid compositions described herein may be formulated for use in treatment of tau-mediated neurodegenerative diseases and conditions.

### Cannabinoid Compositions

In some aspects, provided are cannabinoid compositions comprising a combination of CBD, CBG and THC, which are collectively present as the major components of the cannabinoids in the compositions. In certain embodiments, the compositions herein, including the compositions administered in the methods herein, are drug formulations that comprise a combination of extracts or isolated compounds from one or more cultivars that are blended to achieve certain ratios of CBD, CBG and THC. For example, in some variations, extracts from genetically identical clones of three different cultivars (e.g., high CBD cultivars, high CBG cultivars, and high THC cultivars) may be used to produce the drug formulations. The components of the cannabinoid compositions provided herein are described in further detail below.

In some variations, the CBD, CBG and THC are collectively greater than 50%, greater than 60%, greater than 70%, greater 80%, greater than 85%, greater than 90%, greater than 95%, greater than 96%, greater than 97%, greater than 98%, or greater than 99%; or between 50% and 99.9%, between 60% and 99%, between 70% and 99%, between 80% and 99%, between 85% and 99%, between 85% and 95%, or between 90% and 99% by weight of the cannabinoids present in the composition.

It should be understood that in addition to the cannabinoids, in some embodiments, the cannabinoid compositions may include other cannabinoids as well as non-cannabinoids formulated in a vehicle, such as a lipid vehicle, as described in further detail below. Such other cannabinoids as well as non-cannabinoids are present from the cannabis plant from which the compositions are obtained. Thus, in some variations, the composition comprises CBD, CBG and THC in the ratios and amounts as described herein, as well as other components, such as terpenes, and lipid excipients.

The structures of CBD, CBG and THC are well understood in the art. In some embodiments, the THC present in the compositions herein is primarily in the form of (−)-delta-9-trans-tetrahydrocannabinol (Δ9-THC).

In some variations, the CBD, CBG and THC are present in a molar ratio between about 100:100:1 and about 1:1:1; or between about 100:50:1 and about 20:1:1. In certain variations, the CBD and CBG are present in a molar ratio between about 100:1 and about 1:1. In certain variations, the molar ratio of CBD and THC is between about 50:1 and about 1:1.

In some variations, the CBD, CBG and THC are present in a weight ratio between about 100:100:1 and about 1:1:1; or between about 100:50:1 and about 20:1:1. In certain variations, the CBD, CBG and THC are present in a weight ratio between about 1:0.05-0.42:0.003-0.03. In certain variations, the CBD and CBG are present in a weight ratio between about 100:1 and about 1:1. In certain variations, the CBD and CBG are present in a weight ratio between about 1:0.05-0.42. In certain variations, the weight ratio of CBD and THC is between about 50:1 and about 1:1. In certain variations, the weight ratio of CBD and THC is between about 1:0.003-0.03.

In some variations, the CBD is greater than half of the cannabinoids present in the composition by weight. In certain variations, the CBD is greater than 49% by weight, or between 49% and 98% by weight of the cannabinoids present in the composition. In other variations, the combination of CBD and CBG is greater than half of the cannabinoids present in the composition by weight.

In some variations, the CBD is greater than 5 mg/ml, between about 50 mg/ml and 150 mg/ml, or between about 5 mg/ml and 500 mg/ml in the total composition; and the CBG is between about 7 mg/ml and 21 mg/ml, between about 5 mg/ml and 95 mg/ml, between about 5 mg/ml and 50 mg/ml, or between about 5 mg/ml and 20 mg/ml in the total composition. In a variation of the foregoing, the “total composition” includes cannabinoids, non-cannabinoids (e.g., terpenes, if present), and a vehicle (e.g., lipid vehicle).

It should be understood that any suitable methods and techniques known in the art may be employed to measure the amounts of the components in the compositions. For example, gravimetric or volumetric methods may be employed to quantify the components present in the composition. One of skill in the art would appreciate how to convert the mg/ml units to other suitable units, such as mg/g.

In other variations, the CBD is greater than 1% by weight, or between 1% and 90% by weight of the total composition; and the CBG is greater than 0.3% by weight, or between 0.3% and 49% by weight of the total composition. In a variation of the foregoing, the “total composition” includes cannabinoids, non-cannabinoids (e.g., terpenes, if present), and a vehicle (e.g., lipid vehicle).

In some variations, the THC is present in an amount less than the limit set forth by the appropriate regulatory agencies, including for example, the U.S. Food and Drug Administration (FDA) or the U.S. Drug Enforcement Administration (DEA) or the United States Department of Agriculture (USDA) (e.g., with respect to the 2018 Farm Bill for Hemp products). In certain variations, the THC is less than 0.3% by weight, or between 0.05% and 0.3% by weight of the cannabinoids present in the composition. In certain variations, the THC is less than 2 mg/ml, or between about 0.5 mg/ml and 1.5 mg/ml in the total composition. In a variation of the foregoing, the “total composition” includes cannabinoids, non-cannabinoids (e.g., terpenes, if present), and a vehicle (e.g., lipid vehicle).

In some embodiments, the cannabinoid compositions provided herein further comprise additional components, including other cannabinoids and/or non-cannabinoids. For example, in some variations, the composition further comprises one or more of the following: cannabidiolic acid (CBDA), tetrahydrocannabinolic acid (THCA), cannabichromene (CBC), and terpenes (such as alpha-bisabolol, guaiol, beta-caryophyllene, caryophyllene oxide, alpha-humulene or alpha-caryophyllene, limonene, linalool, beta-myrcene, trans-nerolidol, (E)-b-ocimene, alpha-pinene, beta-pinene, terpineols, terpnolene, and valencene).

In other variations, the composition further comprises one or more of the following: cannabinol (CBN), cannabichromene (CBC), tetrahydrocannabivarin (THCV), cannabigerolic acid (CBGA), cannabichromene acid (CBCA), cannabichromene acid (CBCA), tetrahydrocannabinolic acid (THCA), and cannabidiolic acid (CBDA), or any derivatives thereof.

In other embodiments, the composition may further comprise one or more of the following compounds:
Cannabigerol-type compounds: cannabigerol ((E)-CBG C-5), cannabigerol monomethyl ether ((E)-CBGM C-5A), Cannabinerolsäure A ((Z)-CBGA C-5A), Cannabigerovarin (((e)-BGV C-3), Cannabigerolsäure A (e)-CBGA C-5A), A Cannabigerolsäure monomethyl ether ((e)-CBGAM C-5A), Cannabigerovarinsäure A ((e)-CBGVA-C3A);
Cannabichromene-type compounds: cannabichromene (CBC-C5), Cannabichromensäure A (CBCA C-5A), Cannabichromevarin (CBCVC-3), Cannabichromevarinsäure A (CBCVA-C3A);
Cannabidiol-type compounds: cannabidiol (CBD-C5), cannabidiol monomethyl (CBDM-C5), cannabidiol-C4 (CBD-C4), Cannabidivarin (CBDV-C3), Cannabidiorcol (CBD-C1), cannabidiolic (CBDA C-5), Cannabidivarinsäure (CBDVA C-3);
Cannabinodiol-type compounds: Cannabinodiol (CBND C-5), Cannabinodivarin (CBND C-3);
Tetrahydrocannabinol-type compounds: Δ⁹-tetrahydrocannabinol (Δ⁹-THC-C5), Δ⁹-tetrahydrocannabinol-C4 (Δ9-THC-C4), Δ⁹-tetrahydrocannabivarin (Δ⁹-THCV-C3), Δ⁹-Tetrahydrocannabiorcol (Δ⁹-THCO C-1), Δ⁹-Tetrahydrocannabinolsäure (49 THCA-C-5A), Δ⁹-Tetrahydrocannabinolsäure B (Δ⁹ THCA-C-5B), Δ⁹-Tetrahydrocannabinolsäure-C4(Δ9 THCA-C-4A and/or B), Δ⁹-Tetrahydrocannabivarinsäure A (Δ⁹-THCVA-C3A), Δ⁹-Tetrahydrocannabiorcolsäure (Δ⁹-THCOA-C1 A and/or B), (−)-48-trans-(6aR, 10aR)-8-tetrahydrocannabinol (Δ⁸-THC-C5), (−)-Δ⁸-trans-(6aR, 10aR)-Tetrahydrocannabinolsäure A (Δ⁸-THCA-C 5A); (−)-(6a S, 10a R)-Δ⁹-tetrahydrocannabinol ((−)-cis-Δ⁹-THC-C5);
Cannabinol-type compounds: Cannabinol CBN-C5, cannabinol C4 (CBN-C4), Cannabivarin (CBN-C3), cannabinol C2 (CBN-C2), Cannabiorcol (CBN-C1), Cannabinolsäure A (C5 CBNA-A), Cannabinolmethylether (CBNM C-5);
Cannabitriol-type compounds: (−)-(9R,10R)-trans-Cannabitriol ((−)-trans-CBT-C5), (+)-(9S,10S)-Cannabitriol ((+)-trans-CBT C-5), (+)-(9R, 10S/9S, 10R)-Cannabitriol ((±)-cis-CBT-C5), (−)-(9R,10R)-trans [10-0-thyl-cannabitriol] ((−)-trans-CBT-OEt-C5), (±)-(9R, 10R/9S, 10S)-Cannabitriol-C3 (±)-trans-CBT-C3), 8,9-dihydroxy-Δ6a (10a) tetrahydrocannabinol (8,9-di-OH-CBT-C5), cannabidiolic A (CBDA C-59-OH-CBT-C5 ester), (−)-(6aR, 9S, 10S, 10aR)-9,10-dihydroxy-hexahydrocannabinol, Cannabiripsol Cannabiripsol-C5, (−)-6a,7,10a-trihydroxy-Δ⁹-tetrahydrocannabinol ((−)-Cannabitetrol), 10-oxo-Δ6a (10a) tetrahydrocannabinol (OTHC);
Cannabielsoin-type compounds: (5aS, 6S, 9R, 9aR)-C5-Cannabielsoin (CBEC-5), (5aS, 6S, 9R, 9aR)-C3-Cannabielsoin (CBE C-3), (5aS, 6S, 9R, 9aR)-Cannabielsoinsäure A (CBEA-C5 A), (5aS, 6S, 9R, 9aR)-Cannabielsoinsäure B (CBEA-C5 B), (5aS, 6S, 9R, 9aR)-C3 Cannabielsoinsäure B (CBEA-C3 B), Cannabiglendol-C3 (OH-iso-HHCV C-3), Dehydrocannabifuran (DCBF C-5), Cannabifuran (CBF-C5);
Isocannabinoide-type compounds: (−)-Δ⁷-trans-(1R, 3R, 6R) Isotetrahydrocannabinol, (±)-Δ⁷-1,2-cis-(1R, 3R, 6S/1S, 3S, 6R)-Isotetrahydrocannabivarin, (−)-Δ7-trans-(1R, 3R, 6R)-Isotetrahydrocannabivarin;
Cannabicyclol-type compounds: (±)-(1aS, 3aR, 8bR, 8Cr-cannabicyclol (CBL-C), (±)-(1aS, 3aR, 8bR, 8Cr-Cannabicyclolsäure A (CBLA-C5A) (±)-(1aS, 3aR, 8bR, 8Cr-Cannabicyclovarin (CBLV C-3);
Cannabicitran-type compounds: Cannabicitran (CBT-C5); and
Cannabichromanon-type compounds: Cannabichromanon (CBCN C-5), Cannabichromanon-C3 (CBCN C-3), Cannabicoumaronon (CBCON C-5).

In addition to the above cannabinoids, the carboxylic acids which are biosynthetic precursors of each are contemplated as cannabinoids that may be present in the compositions described herein. In such instances, such cannabinoids are present as a minor component in the composition. In some variations, the cannabinoid precursors are not present in a detectable amount in the composition.

In some variations, minor cannabinoids present are collectively less than about 5% or less than about 2.5% by weight of the total composition. In a variation of the foregoing, the “total composition” includes cannabinoids, non-cannabinoids (e.g., terpenes, if present), and a vehicle (e.g., lipid vehicle).

In other variations, the compositions further comprise terpenes. Examples of terpenes that may be detected in the compositions include, for example, alpha-bisabolol, guaiol, beta-caryophyllene, caryophyllene oxide, alpha-humulene or alpha-caryophyllene, limonene, linalool, beta-myrcene, trans-nerolidol, (E)-b-ocimene, alpha-pinene, beta-pinene, terpineols, terpnolene, and valencenealpha-bisabolol, beta-caryophyllene oxide, and guaiol. In one variation, depending on the source of the cannabinoids as described in further detail below, no detectable amounts of terpenes may be found.

In yet other variations, the composition further comprises flavonoids. In one variation, depending on the source of the cannabinoids as described in further detail below, no detectable amounts of flavonoids may be found.

It should be understood that the minor cannabinoids, terpenes and flavonoids, if present in the composition, may be from the BDS and/or extracts used to provide the CBD, CBG and THC, and such sources are described in further detail below.

### Sources of CBD, CBG and THC

In some embodiments of the cannabinoid compositions provided herein, the CBD, CBG and THC are provided as a combination of extracts or isolated from at least two cannabis cultivars.

In some variations when the compositions comprise a combination of BDS, extracts or blended extracts (as described in further detail below), such compositions are polymodal compositions that include multiple active components that affect multiple targets and implicate multiple mechanisms of action simultaneously. The polymodality of such compositions may positively affect efficacy and safety profile. Such polymodal compositions may be viewed as distinct from fixed dose combinations (“FDCs”) that typically will use highly purified or isolated cannabinoid components.

### BDS

In some embodiments, one or more of CBD, CBG and THC are provided in the form a botanical drug substance (BDS). In some variations, the CBD, CBG and THC are each in the form of BDS. In some variations, a “botanical drug substance” or “BDS” is defined in the Guidance for Industry Botanical Drug Products Draft Guidance, August 2000, US Department of Health and Human Services, Food and Drug Administration Centre for Drug Evaluation and Research as: “A drug derived from one or more plants, algae, or microscopic fungi. It is prepared from botanical raw materials by one or more of the following processes: pulverisation, decoction, expression, aqueous extraction, ethanolic extraction or other similar processes.” A botanical drug substance does not include a highly purified or chemically modified substance derived from natural sources. Thus, in the case of cannabis, BDS derived from cannabis plants do not include highly purified pharmaceutical grade cannabinoids.

### Extracts

In other embodiments, one or more of CBD, CBG and THC are provided as extracts from the cannabis plant. Such extracts may be obtained using any suitable methods and techniques known in the art. For example, dried cannabis flowers are soaked in water or alcohol to obtain the trichomes from the plant. The trichomes undergo solvent extraction and optionally additional purification steps to obtain a cannabinoid-rich oil, also referred to as an “extract”.

In some variations, the CBD, CBG and THC are provided as a combination of cannabis extracts or isolated from 2-4 cannabis cultivars. In certain variations, the CBD, CBG and THC are provided as a combination of cannabis extracts from genetically identical clones of 3 different cannabis cultivars. In one variation for the foregoing, the 3 different cultivars are a high CBD cultivar, a high CBG cultivar and a high THC cultivar. In some variations, the cannabis cultivars are Cannabis sativa cultivars.

In some variations, when the CBD, CBG and THC are provided as extracts, the compositions provided herein further include terpenes. In certain variations, when the CBD, CBG and THC are provided as extracts, the compositions provided herein further include terpenes and flavonoids.

### Blended Extracts

In other embodiments, one or more of the CBD, CBG and THC are provided as a blend of the extracts described above in combination with additional CBD, CBG and/or THC obtained from other sources to achieve the particular ratios and amounts of CBD, CBG and THC as described herein. For example, in some variations, the composition comprises CBD, CBG and THC provided as extracts from the cannabis plants, blended with additional CBD provided in a purified form or as an isolate to achieve the ratios and amounts of CBD, CBG and THC as described herein.

### Purified Forms

In yet other embodiments, one or more of the CBD, CBG and THC are provided in a purified form. Such purified forms of the cannabinoids may be obtained using any suitable methods and techniques known in the art. For example, the extract or blended extracts described above may undergo distillation (e.g., molecular distillation) to remove certain constituents, such as terpenes and lipids, that are non-cannabinoids, and also separate out specific cannabinoids. In some variations, the purified extracts are oils.

In certain variations, the CBD, CBG and THC are a combination of highly purified CBD, highly purified CBG and highly purified THC, wherein each of which are extracted from a cannabis plant and purified to the extent that other cannabinoids and a majority of non-cannabinoid components that are co-extracted with the cannabinoids have been removed. In one variation, the highly purified CBD is greater than or equal to 90% (w/w) pure; the highly purified CBG is greater than or equal to 90% (w/w) pure; and the highly purified THC is greater than or equal to 90% (w/w) pure.

### Isolates

In some variations, one or more CBD, CBG and THC are provided as isolates. Such isolates may be obtained using any suitable methods and techniques known in the art. For example, the isolates may be obtained by crystallization or precipitation of a purified extract as described above to isolate a specific cannabinoid, followed by filtration to yield a powder that is essentially a pure cannabinoid with excess solvent removed. In some variations the isolates are powders. In one variation, the CBD isolate is greater than or equal to 99% (w/w) pure; the CBG isolate is greater than or equal to 99% (w/w) pure; and the THC isolate is greater than or equal to 99% (w/w) pure.

In certain variations, CBD, CBG and THC are provided a combination of isolates, which may be with or without BDS, extracts or blended extracts. In one variation, the CBD, CBG and THC are provided as a combination of isolates and BDS.

Natural vs. Synthetic Sources

In some variations, the CBD, CBG and THC are all naturally derived. In other variations, at least a portion of the CBD, CBG and/or THC is naturally derived, and the other portion is synthetic and/or biosynthetic. Synthetic cannabinoids may include compounds that have a cannabinoid-like structure and are manufactured using chemical processes rather than by the plant. Biosynthetic cannabinoids may include compounds that have a cannabinoid-like structure and are produced using biological processes rather than by the plant. In certain embodiments, at least a portion of CBD present in the composition is prepared synthetically or biosynthetically. In certain embodiments, at least a portion of CBG present in the composition is prepared synthetically or biosynthetically. In certain embodiments, at least a portion of THC present in the composition is prepared synthetically or biosynthetically.

It should be understood that the compositions provided herein may include CBD, CBG and THC provided in a combination of different forms described above. For example, in certain variations, the CBD, CBG and THC are provided in the form of BDS in combination with additional refined or synthetic or biosynthetic CBD, CBG and THC to achieve the ratios and amounts described herein.

### Lipid Vehicles

In some embodiments, the combination of cannabinoids described herein are formulated in lipid vehicles to yield the compositions, e.g., the drug formulation. In some variations, the compositions herein may further comprise at least one lipid excipient. In certain variations, suitable excipients may include glyceryl monolinoleate.

In some variations, the lipid vehicle comprises a winterized oil composed of long-chain mono-, di-, and triglycerides. In certain variations, the lipid vehicle comprises mono-, di- and triglycerides of mainly linoleic (C_{18:2}) and oleic (C_{18:1}) acids. In one variation of the foregoing, the diester fraction is predominant.

In other embodiments, the lipid vehicle comprises self-emulsifying drug delivery systems.

### Other Components

In other embodiments, the compositions provided herein may further include or more additional components. For example, in some variations, the compositions further comprise at least one fatty acid. In certain variations, the compositions further comprise long-chain omega-3 polyunsaturated fatty acids (O-3s). In one variation, the compositions further comprise docosahexaenoic acid (DHA) and eicosapentaenoic acid (EPA).

In some embodiments, the drug substance compositions comprise CBD, CBG and THC, collectively a major component of the cannabinoids present in the composition, in the ratios and amounts as described herein; fats and fatty acids; and terpenes. In certain embodiments, the drug substance consists essentially of CBD, CBG and THC, collectively a major component of the cannabinoids present in the composition, in the ratios and amounts as described herein; fats and fatty acids and terpenes.

In some variations, the drug substance compositions comprise between 70% and 90% cannabinoids, including CBD, CBG and THC, collectively a major component of the cannabinoids present in the composition, in the ratios and amounts as described herein; between 10% and 15% fats and fatty acids; and between 1% and 5% terpenes. For example, in one variation, the composition comprises about 80% cannabinoids in the ratios and amounts as described herein, about 15% fats and fatty acids, and about 5% terpenes. In certain variations, the drug substance compositions consist essentially of between 70% and 90% cannabinoids in the ratios and amounts as described herein; between 10% and 15% fats and fatty acids; and between 1% and 5% terpenes.

In some embodiments, the drug product compositions comprise CBD, CBG and THC, collectively a major component of the cannabinoids present in the composition, in the ratios and amounts as described herein; fats and fatty acids; terpenes; and at least one lipid, such as a winterized oil composed of long-chain mono-, di-, and triglycerides. In certain embodiments, the drug product compositions consist essentially of CBD, CBG and THC, collectively a major component of the cannabinoids present in the composition, in the ratios and amounts as described herein; fats and fatty acids; terpenes; and at least one lipid, such as a winterized oil composed of long-chain mono-, di-, and triglycerides.

### Preparation Methods

The cannabinoid compositions provided herein may be obtained from combining plant-derived, synthetic and/or biosynthetic CBD, CBG and THC, in order to achieve the appropriate amounts and ratios of these components.

As discussed above, when CBD, CBG and THC are plant-derived, they may be obtained from a cannabis plant. Various methods, techniques and conditions to cultivate, harvest and process cannabis plants are generally known in the art. Further, the resulting cannabis extract may be characterized using methods known in the art. Any suitable processes known in the art may be employed to obtain the CBD, CBG and THC used herein.

For example, bulk plant material is isolated from dried cannabis flower. The bulk plant material is separated from the botanical starting material. The botanical starting materials are weighed and stored in an amber jar. The botanical starting material are added to an extraction vessel with solvent. The solvent is removed via vacuum distillation until only refined cannabis oil is present, with a low solvent concentration. The crude cannabis oil is then heated to for a suitable time to convert the THCA to THC to yield a refined cannabis oil. The main cannabinoids, THCA, CBDA and CBGA are converted to the base molecule THC, CBD and CBG, respectively.

The cannabinoid extracts described above may undergo further purification using methods and techniques known in the art to obtain purified extracts or isolates. The purified extracts are typically in oil form, whereas the isolates are typically in powder form. In some variations, cannabis extracts may undergo distillation to remove certain constituents, such as terpenes and lipids, that are non-cannabinoids, and also separate out specific cannabinoids to yield a purified extract. In other variations, such purified extract may undergo crystallization or precipitation to isolate a specific cannabinoid, followed by filtration to yield a powder that is essentially a pure cannabinoid with excess solvent removed.

The cannabinoid compositions provided herein are pharmaceutical cannabinoid compositions, formulated based on the mode of intended administration. For example, in some embodiments, administration may be ocular, oral, parenteral, topical, etc. In one variation, the cannabinoid composition is formulated for oral administration. In some embodiments, the cannabinoid compositions may be formulated with one or more excipients to increase stability, increase shelf-life, or increase efficacy. Cannabinoid compositions disclosed herein may be formulated for administration according to methods known in the art.

### Treatment Methods

In some aspects, provided is a method for treating neurodegenerative diseases and conditions in a subject.

In some embodiments, the neurodegenerative diseases and conditions involve tau-mediated neurodegeneration. In certain embodiments, the neurodegenerative diseases and conditions are associated with neuronal insult, for example, characterized by intracellular aggregates of the microtubule associated protein Tau. In certain embodiments, the methods for treating neurodegenerative diseases and conditions reduce (or partially reduces) toxic gains of function, and/or restores (or partially restores) or reduces (or partially reduces) normal Tau function in the subject.

Thus, in certain aspects, provided is a method for treating neurodegenerative diseases and conditions in a subject, comprising: a) administering a cannabinoid composition as disclosed herein; and b) reducing (or partially reducing) toxic gains of function, and/or restoring (or partially restoring) or reducing (or partially reducing) normal Tau function in the subject.

In some embodiments, the neurodegenerative diseases and conditions may include Alzheimer's disease, Pick's disease, progressive supranuclear palsy, frontotemporal dementia, epilepsy, concussion, stroke, and traumatic brain injury (TBI).

In one aspect, provided is a method for treating cancer in in a subject in need thereof. In some variations, the method targets Tau protein in the treatment of the cancer.

In other aspects, provided is a method for treating diseases and conditions related to aging.

In yet other aspects, provided are methods to treat neurological disorders and conditions, pain diseases and conditions, and urologic diseases and conditions. In some embodiments, the neurological disorders may include restless leg syndrome (RLS), Alzheimer's Dementia (AD), and Post-Traumatic Epilepsy. In some embodiments, pain diseases and conditions may include Arthritis, Rheumatoid Arthritis, Aromatase Inhibitor Induced Arthralgia, and Complex Regional Pain Syndrome. In some embodiments, the urologic diseases and conditions, such as nocturia. In some embodiments, the nocturia results from benign prostatic hyperplasia.

In other aspects, provided is a method for treating tau-mediated diseases and conditions in a subject. In certain embodiments, the diseases and conditions are characterized by intracellular aggregates of the microtubule associated protein Tau. In certain embodiments, the methods for treating diseases and conditions reduce (or partially reduces) toxic gains of function, and/or restores (or partially restores) or reduces (or partially reduces) normal Tau function in the subject.

In some embodiments of the foregoing aspects, the method comprises administering to the subject the compositions described herein, e.g., comprising CBD, CBG and THC as the major components therein. In some variations of the foregoing aspects, the terms “treating” or “treatment”, as used herein, refer to a method or procedure for obtaining beneficial or desired results—for example, clinical results. Beneficial or desired results may include: (1) alleviating one or more symptoms caused by or associated with a disease, disorder, or condition; (2) reducing the extent of the disease, disorder, or condition; (3) slowing or stopping the development or progression of one or more symptoms caused by or associated with the disease, disorder, or condition (for example, stabilizing the disease, disorder, or condition); and (4) relieving the disease, for example, by causing the regression of one or more clinical symptoms (e.g., ameliorating the disease state, enhancing the effect of another medication, delaying or stopping the progression of the disease, increasing the quality of life, and/or prolonging survival rates). Indications-broad categories, specific examples etc.

In some variations of the foregoing aspects, the term “therapeutically effective amount” applied to dose or amount refers to that quantity of a composition or formulation, such as those described herein, that is sufficient to result in a desired clinical benefit after administration to a subject in need thereof. It is to be understood that the amount may be in one or more doses, e.g., a single dose or multiple doses may be needed to achieve the desired treatment endpoint.

In some variations of the foregoing aspects, the subject is a human. In one variation, the subject is an adult human.

### Kits and Articles of Manufacture

In other aspects, the present disclosure further provides kits for carrying out the methods of the invention. The kits may comprise the cannabinoid compositions described herein and suitable packaging. In some embodiments, provided is a kit, comprising: (i) a therapeutically effective amount of any of the cannabinoid compositions described herein; and (ii) a label and/or instructions for use in treating any of the indications described herein, including neurodegenerative diseases and conditions, as well as cancer.

In yet other aspects, the present disclosure further provides an article of manufacture, comprising a therapeutically effective amount of any of the cannabinoid compositions described herein in a suitable container.

### ENUMERATED EMBODIMENTS

The following enumerated embodiments are representative of some aspects of the invention.
1. A method for treating a neurodegenerative disease or condition, or a cancer in a subject in need thereof, comprising: administering to the subject a therapeutically effective amount of a cannabinoid composition,
   wherein the cannabinoid composition comprises cannabidiol (CBD), cannabigerol (CBG), and tetrahydrocannabinol (THC) present as a major component therein,
   wherein the CBD, THC/or CBG are isolated or purified.
2. The method of embodiment 1, wherein the CBD, CBG and/or THC are in the form of a botanical drug substance.
3. The method of embodiment 1, wherein one or more of CBD, CBG, and THC are in the form of an extract or isolated compounds produced from one or more cultivars that are blended together.
4. The method of any one of embodiments 1 to 3, wherein the CBD, CBG and THC are present in a molar ratio between about 100:100:1 and about 1:1:1.
5. The method of embodiment 4, wherein the molar ratio is between about 100:50:1 and about 20:1:1.
6. The method of any one of embodiments 1 to 5, wherein the cannabinoid composition further comprises at least one lipid excipient.
7. The method of embodiment 6, wherein at least one lipid excipient is a winterized oil comprising long-chain mono-, di-, and triglycerides.
8. The method of any one of embodiments 1 to 7, wherein the composition further comprises docosahexaenoic acid (DHA), or eicosapentaenoic acid (EPA), or a combination thereof.
9. The method of any one of embodiments 1 to 8, wherein the neurodegenerative disease or condition is associated with neuronal insult.
10. The method of embodiment 9, wherein the neuronal insult is characterized by intracellular aggregates of microtubule associated protein Tau.
11. The method of any one of embodiments 1 to 8, wherein the neurodegenerative disease or condition is Alzheimer's disease, Pick's disease, progressive supranuclear palsy, frontotemporal dementia, epilepsy, concussion, stroke, or traumatic brain injury.
12. The method of any one of embodiments 1 to 11, wherein the method targets microtubule associated protein Tau.
13. A method for treating a tau-mediated disease or condition in a subject in need thereof, comprising: administering to the subject a therapeutically effective amount of a cannabinoid composition,
   wherein the cannabinoid composition comprises cannabidiol (CBD), cannabigerol (CBG), and tetrahydrocannabinol (THC) present as a major component therein,
   wherein the CBD, THC/or CBG are isolated or purified.
14. The method of embodiment 13, wherein the disease or condition is characterized by intracellular aggregates of the microtubule associated protein Tau.
15. The method of 13 or 14, wherein the method reduces or partially reduces toxic gains of function, and/or restores or partially restores or reduces (or partially reduces normal Tau function in the subject.
16. A cannabinoid composition, comprising:
   cannabidiol (CBD), cannabigerol (CBG), and tetrahydrocannabinol (THC) present as a major component therein,
   wherein the CBD, THC/or CBG are isolated or purified.
17. The composition of embodiment 16, wherein the CBD, CBG and/or THC are in the form of a botanical drug substance.
18. The composition of embodiment 16 or 17, wherein one or more of CBD, CBG, and THC are in the form of an extract or isolated compounds produced from one or more cultivars that are blended together,
19. The composition of any one of embodiments 16 to 18, wherein the CBD, CBG and THC are present in a molar ratio between about 100:100:1 and about 1:1:1.
20. The composition of embodiment 19, wherein the molar ratio is between about 100:50:1 and about 20:1:1.
21. The composition of any one of embodiments 16 to 20, wherein the cannabinoid composition further comprises at least one lipid excipient.
22. The composition of embodiment 21, wherein at least one lipid excipient is a winterized oil comprising long-chain mono-, di-, and triglycerides.
23. The composition of any one of embodiments 16 to 22, wherein the composition further comprises docosahexaenoic acid (DHA), or eicosapentaenoic acid (EPA), or a combination thereof.
24. A cannabinoid composition, comprising:
cannabidiol (CBD), cannabigerol (CBG), and tetrahydrocannabinol (THC), collectively a major component of the cannabinoids present in the composition,
wherein the CBD, CBG and THC are provided as a combination of extracts or isolated from at least two cannabis cultivars.
25. The composition of embodiment 24, wherein the CBD, CBG and/or THC are provided as botanical drug substances (BDS).
26. The composition of embodiment 25, further comprising other cannabinoid and/or non-cannabinoid components that are present from the BDS.
27. The composition of embodiment 25, further comprising terpenes that are present from the BDS.
28. The composition of any one of embodiments 24 to 27, further comprising flavonoids that are present from the BDS.
29. The composition of embodiment 24, wherein the CBD, CBG and THC are provided as a combination of cannabis extracts.
30. The composition of embodiment 24, wherein the CBD, CBG and THC are provided as a combination of extracts from 2-4 cannabis cultivars.
31. The composition of embodiment 24, wherein the CBD, CBG and THC are provided as a combination of extracts from genetically identical clones of 3 different cannabis cultivars.
32. The composition of embodiment 31, wherein the 3 different cannabis cultivars are a high CBD cultivar, a high CBG cultivar and a high THC cultivar.
33. The composition of any one of embodiments 30 to 32, wherein cannabis cultivars are Cannabis sativa cultivars.
34. The composition of any one of embodiments 29 to 33, further comprising other cannabinoid and/or non-cannabinoid components that are present from the extracts.
35. The composition of any one of embodiments 29 to 33, further comprising terpenes that are present from the extracts.
36. The composition of any one of embodiments 29 to 35, further comprising flavonoids that are present from the extracts.
37. The composition of any one of embodiments 25 to 36, further comprising additional CBD, CBG and/or THC provided in purified form.
38. The composition of any one of embodiments 25 to 36, further comprising additional CBD, CBG and/or THC isolates.
39. The composition of embodiment 24, wherein the CBD, CBG and THC are provided as a combination of purified CBD, CBG and THC cannabis extracts.
40. The composition of embodiment 24, wherein the CBD, CBG and THC are provided as a combination of CBD, CBG and THC isolates.
41. The composition of embodiment 24, wherein the CBD, CBG and THC are provided as a combination of BDS and CBD, CBG and/or THC isolates.
42. The composition of embodiment 24, wherein the CBD, CBG and THC are provided as a combination of BDS, and purified CBD, CBG and/or THC cannabis extracts.
43. The composition of embodiment 24, wherein the CBD, CBG and THC are provided as a combination of BDS, and CBD, CBG and/or THC isolates.
44. The composition of embodiment 24, wherein the CBD, CBG and THC are provided as BDS in combination with refined or synthetic CBD, CBG and/or THC.
45. The composition of embodiment 24, wherein the CBD, CBG and THC are a combination of highly purified CBD, highly purified CBG and highly purified THC, wherein each of which are extracted from a cannabis plant and purified to the extent that other cannabinoids and a majority of non-cannabinoid components that are co-extracted with the cannabinoids have been removed.
46. The composition of embodiment 45, wherein the highly purified CBD is greater than or equal to 90% (w/w) pure; the highly purified CBG is greater than or equal to 90% (w/w) pure; and the highly purified THC is greater than or equal to 90% (w/w) pure.
47. The composition of embodiment 24, wherein the CBD, CBG and THC are all naturally derived.
48. The composition of embodiment 24, wherein at least a portion of the CBD, CBG and/or THC is naturally derived, and the other portion is synthetic and/or biosynthetic.
49. The composition of any one of the preceding embodiments, wherein the THC is present primarily in the form of (−)-delta-9-trans-tetrahydrocannabinol (Δ9-THC).
50. The composition of any one of the preceding embodiments, wherein the CBD, CBG and THC are collectively greater than 50% by weight of the cannabinoids present in the composition.
51. The composition of embodiment 50, wherein the CBD, CBG and THC are collectively greater than 60% by weight of the cannabinoids present in the composition.
52. The composition of any one of the preceding embodiments, wherein the CBD, CBG and THC are present in a molar ratio between about 100:100:1 and about 1:1:1.
53. The composition of embodiment 52, wherein the CBD, CBG and THC are present in a molar ratio is between about 100:50:1 and about 20:1:1.
54. The composition of any one of the preceding embodiments, wherein the CBD and CBG are present in a molar ratio between about 100:1 and about 1:1.
55. The composition of any one of the preceding embodiments, wherein the molar ratio of CBD and THC is between about 50:1 and about 1:1.
56. The composition of any one of the preceding embodiments, further comprising at least one lipid excipient.
57. The composition of embodiment 56, wherein at least one lipid excipient is a winterized oil comprising long-chain mono-, di-, and triglycerides.
58. The composition of any one of the preceding embodiments, further comprising docosahexaenoic acid (DHA), or eicosapentaenoic acid (EPA), or a combination thereof.
59. The composition of any one of the preceding embodiments, wherein the composition is formulated for oral delivery.
60. A method for treating a neurodegenerative disease or condition, or a cancer in a subject in need thereof, comprising: administering to the subject a therapeutically effective amount of a cannabinoid composition of any one of embodiments 1 to 36.
61. The method of embodiment 60, wherein the neurodegenerative disease or condition is associated with neuronal insult.
62. The method of embodiment 61, wherein the neuronal insult is characterized by intracellular aggregates of microtubule associated protein Tau.
63. The method of any one of embodiments 60 to 62, wherein the neurodegenerative disease or condition is Alzheimer's disease, Pick's disease, progressive supranuclear palsy, frontotemporal dementia, epilepsy, concussion, stroke, or traumatic brain injury.
64. The method of any one of embodiments 60 to 63, wherein the method targets microtubule associated protein Tau.
65. The method of any one of embodiments 60 to 63, further comprising: targeting microtubule associated protein Tau.
66. A method for treating a tau-mediated disease or condition in a subject in need thereof, comprising: administering to the subject a therapeutically effective amount of a cannabinoid composition of any one of embodiments 24 to 59.
67. The method of embodiment 66, wherein the disease or condition is characterized by intracellular aggregates of the microtubule associated protein Tau.
68. The method of 66 or 67, wherein the treatment at least partially reduces toxic gains of function, and/or at least partially restores or at least partially reduces normal Tau function in the subject.
69. The method of 66 or 67, further comprising: at least partially reducing toxic gains of function, and/or at least partially restoring or at least partially reducing normal Tau function in the subject.

### EXAMPLES

The presently disclosed subject matter will be better understood by reference to the following Examples, which are provided as exemplary of the invention, and not by way of limitation.

### Example 1

### Extraction and Isolation of Cannabinoids from the Plant

A sleeve was inserted into the extraction chamber of a supercritical CO2 extractor. Ground, dried and cured cannabis flower was packed inside the sleeve, with tamping for compaction. Plant material was extracted. Terpenes were collected and stored at −20 C. Cannabinoids were extracted at a suitable pressure and temperature, and dissolved in ethanol. Alternatively, fresh frozen plant material was extracted with warm, absolute, non-denatured ethanol, with homogenation, then filtered. This solution was placed at −25° C. for 24 h or more, then filtered to remove non-cannabinoid fates and lipids. The resulting filtrate was returned to the freezer for 24 h then filtered again. The ethanol-containing cannabinoids was processed in a rotary evaporator to remove the ethanol. The resulting cannabinoid oil was then decarboxylated for a suitable amount of time. The resulting “refined oil” is analyzed and stored at −20 C.

### Example 2

### Effect of Cannabis Extracts Containing Cannabinoids on Lifespan and Healthspan in the Preclinical Tau C. elegans Model

Many neurodegenerative diseases and conditions associated with neuronal insult are characterized by intracellular aggregates of the microtubule associated protein Tau. The recent development and validation of the BR5270 transgenic Caenorhabditis elegans strain exhibits accelerated Tau aggregation, severely impaired motility as a result of neuronal dysfunction, and a shortened lifespan compared to wild-type controls. Due to the significant impact on lifespan and locomotion defect produced by this model, time/cost-effective and high throughput acquisition of data, the C. elegans model of tauopathy serves as an advantageous screening tool for the discovery of novel modulators of neurodegeneration.

### Materials:

Cannabinoid botanical drug substances (20 μM, 40 μM, 80 μM) were prepared in accordance with the protocol described in Example 1 above. The exemplary cannabinoid botanical drug substance used in the study of this example includes CBD, CBG and THC present as the major components in a ratio of 50:20:1.

### Methods:

The effect of the two different complex botanical mixtures described above were compared to the comparative control, cannabidiol (40 μM) and a no treatment negative control in the BR5270 transgenic strain. Additional no-treatment negative control and healthspan extension positive control (cannabidiol 40 μM) in wild-type arms served as added comparators. All examinations were performed in triplicate. The impact of three doses of each compound on animal survival and activity levels were examined on days 4, 8, 12, and 16 of animal life. These time points represent the first day of adulthood, early-, mid-, and late-life periods in the C. elegans lifespan.

Results: Cannabinoid BDS were observed to be more protective than CBD at all three doses tested. See Table 1a below. In Table 1a below, “C₃” refers to Cannabinoid BDS, and “CBD” refers to CBD alone. Survival of C. elegans based on administration of Cannabinoid BDS alone is summarized in Table 1b below. In the table below, Cannabinoid BDS is labeled “BDS”, and “CBD” refers to CBD alone. Overall, the data indicates that Cannabinoid BDS improves longevity and does not lead to a decrease in lifespan (e.g., the formulation is not toxic), and is superior to administration of CBD alone.

TABLE la

| BR5270 | | BR5270 40 μM | | BR5270 80 μM | | BR5270 40 μM | | BR5270 20 μM | |
|---|---|---|---|---|---|---|---|---|---|
| Control | | CBD | | C3 | | C3 | | C3 | |
| % Alive | STD | % Alive | STD | % Alive | STD | % Alive | STD | % | STD |
| 100 | 0 | 100 | 0 | 100 | 0 | 100 | 0 | 100 | 0 |
| 73.43 | 3.155 | 73.17 | 10.38 | 91.97 | 5.589 | 80.39 | 7.336 | 82.11 | 7.411 |
| 38.88 | 3.291 | 39.39 | 4.929 | 52.51 | 3.609 | 54.07 | 8.066 | 49.26 | 7.69 |
| 2.893 | 3.351 | 9.738 | 4.81 | 22.18 | 5.793 | 19.07 | 5.534 | 11.11 | 10.31 |

TABLE 1b

| | | 40 μM | 80 μM | 40 μM | 20 μM |
|---|---|---|---|---|---|
| | Control | CBD | BDS | BDS | BDS |
| Day 12 | 39 | 39 | 52 | 54 | 49 |
| Day 16 | 4 | 10 | 22 | 19 | 11 |

### Example 3A

### Extraction and Isolation of Cannabinoids from the Plant

Bulk plant material was isolated from dried cannabis flower. The bulk plant material was separated from the botanical starting material. The botanical starting materials were weighed and stored in an amber jar. The botanical starting material were added to an extraction vessel with solvent. The solvent was removed via vacuum distillation until only refined cannabis oil was present, with a low solvent concentration. The crude cannabis oil was then heated to for a suitable time to convert the THCA to THC to yield a refined cannabis oil. The main cannabinoids, THCA, CBDA and CBGA were converted to the base molecule THC, CBD and CBG, respectively.

### Example 3B

### Characterization of Cannabinoid Compositions

Exemplary cannabinoid compositions were produced according to Example 3B above, blended with botanical isolates to arrive at the amounts and ratios of CBD, CBG and THC as set forth in Table 2 below, and combined with an oil containing long chain mono, di, and triglycerides as the lipid vehicle. The following Table 2 provides the profile of exemplary drug product compositions, characterized based on cannabinoid content. The compositions were characterized using methods and techniques known in the art, including ultra-performance liquid chromatography (UPLC).

TABLE 2

Exemplary Drug
Product Composition

| | mg/mL | Ratio |
|---|---|---|
| CBD | 50-150 | 1.00 |
| CBG | 7-21 | 0.05-0.42 |
| THC | 0.5-1.5 | 0.003-0.03 |

What is claimed is:

1. A cannabinoid composition, comprising:
cannabidiol (CBD), cannabigerol (CBG), and tetrahydrocannabinol (THC), collectively a major component of the cannabinoids present in the composition,
wherein the CBD, CBG and THC are provided as a combination of extracts or isolated from at least two cannabis cultivars.

2. The composition of claim 1, wherein the CBD, CBG and/or THC are provided as botanical drug substances (BDS).

3. The composition of claim 2, further comprising other cannabinoid and/or non-cannabinoid components that are present from the BDS.

4. The composition of claim 2, further comprising terpenes that are present from the BDS.

5. The composition of any one of claims 2 to 4, further comprising flavonoids that are present from the BDS.

6. The composition of claim 1, wherein the CBD, CBG and THC are provided as a combination of cannabis extracts.

7. The composition of claim 1, wherein the CBD, CBG and THC are provided as a combination of extracts from 2-4 cannabis cultivars.

8. The composition of claim 1, wherein the CBD, CBG and THC are provided as a combination of extracts from genetically identical clones of 3 different cannabis cultivars.

9. The composition of claim 8, wherein the 3 different cannabis cultivars are a high CBD cultivar, a high CBG cultivar and a high THC cultivar.

10. The composition of any one of claims 7 to 9, wherein cannabis cultivars are Cannabis sativa cultivars.

11. The composition of any one of claims 6 to 10, further comprising other cannabinoid and/or non-cannabinoid components that are present from the extracts.

12. The composition of any one of claims 6 to 10, further comprising terpenes that are present from the extracts.

13. The composition of any one of claims 6 to 11, further comprising flavonoids that are present from the extracts.

14. The composition of any one of claims 2 to 13, further comprising additional CBD, CBG and/or THC provided in purified form.

15. The composition of any one of claims 2 to 13, further comprising additional CBD, CBG and/or THC isolates.

16. The composition of claim 1, wherein the CBD, CBG and THC are provided as a combination of purified CBD, CBG and THC cannabis extracts.

17. The composition of claim 1, wherein the CBD, CBG and THC are provided as a combination of CBD, CBG and THC isolates.

18. The composition of claim 1, wherein the CBD, CBG and THC are provided as a combination of BDS and CBD, CBG and/or THC isolates.

19. The composition of claim 1, wherein the CBD, CBG and THC are provided as a combination of BDS, and purified CBD, CBG and/or THC cannabis extracts.

20. The composition of claim 1, wherein the CBD, CBG and THC are provided as a combination of BDS, and CBD, CBG and/or THC isolates.

21. The composition of claim 1, wherein the CBD, CBG and THC are provided as BDS in combination with refined or synthetic CBD, CBG and/or THC.

22. The composition of claim 1, wherein the CBD, CBG and THC are a combination of highly purified CBD, highly purified CBG and highly purified THC, wherein each of which are extracted from a cannabis plant and purified to the extent that other cannabinoids and a majority of non-cannabinoid components that are co-extracted with the cannabinoids have been removed.

23. The composition of claim 22, wherein the highly purified CBD is greater than or equal to 90% (w/w) pure; the highly purified CBG is greater than or equal to 90% (w/w) pure; and the highly purified THC is greater than or equal to 90% (w/w) pure.

24. The composition of claim 1, wherein the CBD, CBG and THC are all naturally derived.

25. The composition of claim 1, wherein at least a portion of the CBD, CBG and/or THC is naturally derived, and the other portion is synthetic and/or biosynthetic.

26. The composition of any one of the preceding claims, wherein the THC is present primarily in the form of (−)-delta-9-trans-tetrahydrocannabinol (Δ9-THC).

27. The composition of any one of the preceding claims, wherein the CBD, CBG and THC are collectively greater than 50% by weight of the cannabinoids present in the composition.

28. The composition of claim 27, wherein the CBD, CBG and THC are collectively greater than 60% by weight of the cannabinoids present in the composition.

29. The composition of any one of the preceding claims, wherein the CBD, CBG and THC are present in a molar ratio between about 100:100:1 and about 1:1:1.

30. The composition of claim 29, wherein the CBD, CBG and THC are present in a molar ratio is between about 100:50:1 and about 20:1:1.

31. The composition of any one of the preceding claims, wherein the CBD and CBG are present in a molar ratio between about 100:1 and about 1:1.

32. The composition of any one of the preceding claims, wherein the molar ratio of CBD and THC is between about 50:1 and about 1:1.

33. The composition of any one of the preceding claims, further comprising at least one lipid excipient.

34. The composition of claim 33, wherein at least one lipid excipient is a winterized oil comprising long-chain mono-, di-, and triglycerides.

35. The composition of any one of the preceding claims, further comprising docosahexaenoic acid (DHA), or eicosapentaenoic acid (EPA), or a combination thereof.

36. The composition of any one of the preceding claims, wherein the composition is formulated for oral delivery.

37. A method for treating a neurodegenerative disease or condition, or a cancer in a subject in need thereof, comprising: administering to the subject a therapeutically effective amount of a cannabinoid composition of any one of claims 1 to 36.

38. The method of claim 37, wherein the neurodegenerative disease or condition is associated with neuronal insult.

39. The method of claim 38, wherein the neuronal insult is characterized by intracellular aggregates of microtubule associated protein Tau.

40. The method of any one of claims 37 to 39, wherein the neurodegenerative disease or condition is Alzheimer's disease, Pick's disease, progressive supranuclear palsy, frontotemporal dementia, epilepsy, concussion, stroke, or traumatic brain injury.

41. The method of any one of claims 37 to 40, wherein the method targets microtubule associated protein Tau.

42. A method for treating a tau-mediated disease or condition in a subject in need thereof, comprising: administering to the subject a therapeutically effective amount of a cannabinoid composition of any one of claims 1 to 36.

43. The method of claim 42, wherein the disease or condition is characterized by intracellular aggregates of the microtubule associated protein Tau.

44. The method of 42 or 43, wherein the treatment at least partially reduces toxic gains of function, and/or at least partially restores or at least partially reduces normal Tau function in the subject.

45. The method of any one of claims 37 to 44, wherein the subject is a human.
